(19) United States
(12) Patent Application Publication
(10) Pub. No.: US 2022/0365015 A1
(43) Pub. Date: Nov. 17, 2022

(54) MIP SENSOR DEVICE WITH REPLACEABLE MIP SENSOR

(71) Applicant: Rhythmic Health, Inc., San Francisco, CA (US)

(72) Inventors: Nicholas Alexander Melosh, Menlo Park, CA (US); Marc Daniel Ferro, Oakland, CA (US); Benjamin Roth Lowenstein, San Francisco, CA (US); Paul Michael Litvak, San Francisco, CA (US)

(21) Appl. No.: 17/322,348

(22) Filed: May 17, 2021

(51) Int. Cl. G01N 27/02; G01N 33/92; G01N 33/74

(52) U.S. Cl. CPC G01N 27/028; G01N 33/92; G01N 33/743; G01N 2600/00

(57) ABSTRACT

A multi-analyte sensor device is disclosed. The multi-analyte device is a handheld device that includes a replaceable sensor. In one embodiment, the replaceable sensor includes a plurality of electrodes coated with one or more different molecular imprinted polymer (MIP) coatings for measuring concentrations of target analytes. The replaceable sensor is configured to be attached or detached from the device one or more times.

### BACKGROUND

### Field of Technology

The subject matter described generally relates to a monolithic molecularly imprinted polymer (MIP) sensor for multiple analytes and, in particular, to a handheld MIP sensor device with a replaceable MIP sensor.

### Discussion of the Related Art

Personalized medicine aims to provide medical diagnostics and treatment to people based on their individual characteristics. In pursuit of this goal, a range of devices and sensors have been developed that enable people to collect physiological data at home without the need for a medical professional to be present. Many households also now have reliable, high-speed connections to the internet, allowing this data to be provided to a remote facility for analysis almost immediately, and the results of that analysis can be returned just as fast. According to some projections, personalized medicine will become a trillion-dollar industry in the next few years. Thus, there is significant demand for low-cost, convenient ways for people to collect physiological data.

### SUMMARY

A handheld multi-analyte sensing device is disclosed. The multi-analyte sensing device measures the concentration of two or more analytes in a biological sample from a user using a multi-analyte sensor. In one embodiment, the multi-analyte sensor is replaceable. The multi-analyte sensing device includes a body and a controller housed within the body. The multi-analyte sensor is configured to be attached and detached from the controller. The multi-analyte sensor may be detached from multi-analyte sensing device in order to replace the multi-analyte sensor with another multi-analyte sensor or to reconfigure the multi-analyte sensor for measuring different analytes.

In one embodiment, the multi-analyte sensor includes a plurality of masks stacked on the multi-analyte sensor. Each mask exposes a specific set of analyte electrodes that are used to sense the concentration of analytes in a biological sample. In one embodiment, the analytes sensed using each mask may be different analytes. Alternatively, the analytes sensed using each mask may be the same allowing for measurements of the same analytes over a period of time.

The multi-analyte sensor may be used to take a plurality of measurements where each measurement is performed using a different one of the masks. After completion of a measurement, the multi-analyte sensor is detached from the controller and the topmost mask is removed from the multi-analyte sensor. The multi-analyte sensor is then re-attached to the device. By removing the topmost mask, another mask is revealed which exposes another set of analyte electrodes that are used to sense the concentration of analytes in a biological sample.

In one embodiment, the case of the multi-analyte sensor device is waterproof. The case may include one or more gaskets that prevent or at least reduce the exposure of the controller to substances that may degrade the controller. The gaskets may be located at the perimeter of the case as well as in an opening in the case through which the multi-analyte sensor attaches to the controller.

### BRIEF DESCRIPTION OF THE DRAWINGS

FIG. 1 is a block diagram of a networked computing environment including a multi-analyte sensing device with a multi-analyte sensor, according to one embodiment.

FIG. 2 illustrates the multi-analyte sensor of the multi-analyte sensing device according to one embodiment.

FIG. 3A illustrates a protective film and FIG. 3B illustrates the protective film on the multi-analyte sensor according to one embodiment.

FIGS. 4A, 4B, and 4C illustrate a plurality of protective films, according to one embodiment.

FIGS. 4D and 4E illustrate the plurality of protective films on the multi-analyte sensor according to one embodiment.

FIG. 5 illustrates the multi-analyte sensing device, according to one embodiment.

FIGS. 6A and 6B illustrate a connection between the multi-analyte sensor and a controller of the multi-analyte sensing device, according to one embodiment.

FIG. 7A illustrates components of the multi-analyte sensing device including sealing mechanism, according to one embodiment.

FIGS. 7B and 7C illustrate a detailed view of the sealing mechanism, according to one embodiment.

FIGS. 8A and 8B illustrate an example approach for calibrating the multi-analyte sensor according to one embodiment.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### System Environment

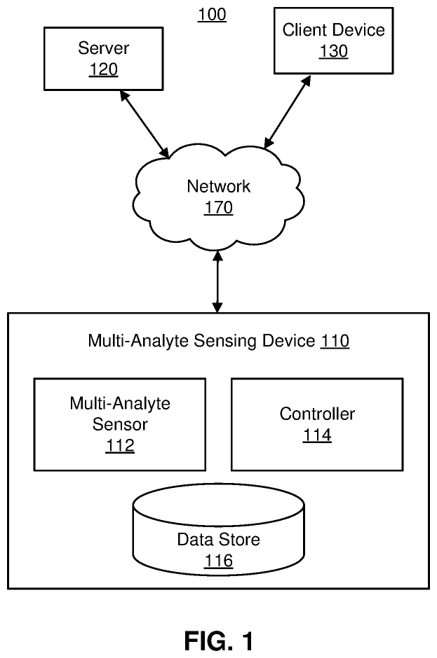

FIG. 1 illustrates one embodiment of a networked computing environment 100. In the embodiment shown, the networked computing environment 100 includes a multi-analyte sensing device 110, a server 120, and a client device 130, all connected via a network 170. In other embodiments, the networked computing environment 100 includes different or additional elements. In addition, the functions may be distributed among the elements in a different manner than described. For example, in some embodiments, the multi-analyte sensing device 110 may not have network connectivity and the server 120, client device 130, and network 170 may be omitted.

The multi-analyte sensing device 110 measures the concentration of two or more analytes in a biological sample from a user. The multi-analyte sensing device 110 may obtain the sample by exposing a multi-analyte sensor 112 included in the device 110 to the sample. In one embodiment, the sample may be saliva, sweat, blood, or urine.

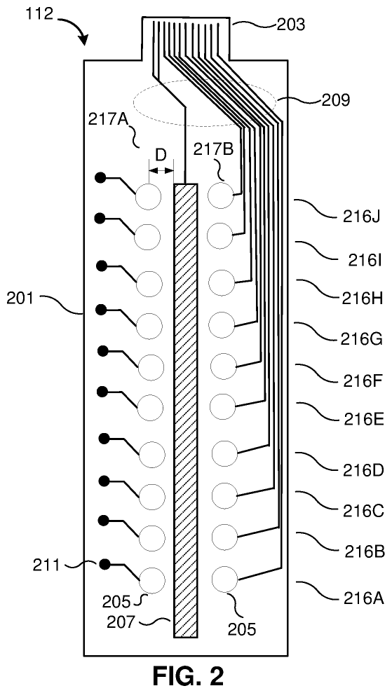

In the embodiment shown in FIG. 1, the multi-analyte sensing device 110 includes the multi-analyte sensor 112, a controller 114, and a data store 116. The multi-analyte sensor 112 is a sensor configured to measure the concentration of two or more analytes in biological samples to which it is exposed. Embodiments of the multi-analyte sensor 112 are described in greater detail below, with reference to FIGS. 2 through 4. The controller 114 includes a processor or other circuitry that receives and processes signals from the multi-analyte sensor 112. The controller 114 may store measurements derived from the signals received from the multi-analyte sensor 112 in the data store 116 or send them via the network 170 to the server 120 or client device 130.

The server 120 and client device 130 are computer systems that may store and analyze measurements provided by the multi-analyte sensing device 110. In one embodiment, the server 120 receives measurements of analyte concentrations from the multi-analyte sensing device 110 and tracks variations in the concentrations over time. The server 120 correlates the variations with one or more health conditions and provides information regarding those health conditions to the client device 130 for display to the user. For example, a sudden spike in cortisol levels indicates acute stress and the user may be advised to undertake relaxation exercises (e.g., deep breathing) whereas consistently high level of cortisol indicates chronic stress and the user might be advised to consider a dietary supplement such as ashguawanda or a lifestyle change.

The network 170 provides the communication channels via which the other elements of the networked computing environment 100 communicate. The network 170 can include any combination of local area and/or wide area networks, using both wired and/or wireless communication systems. In one embodiment, the network 170 uses standard communications technologies and/or protocols. For example, the network 170 can include communication links using technologies such as Ethernet, 802.11, worldwide interoperability for microwave access (WiMAX), 3G, 4G, 5G, Bluetooth, Bluetooth Low Energy (BLE), Long Range Radio (LoRa), code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In some embodiments, all or some of the communication links of the network 170 may be encrypted using any suitable technique or techniques.

### Multi-Analyte Sensors

FIG. 2 is a plan view of one embodiment of the multi-analyte sensor 112. FIG. 2 and is not to scale but rather is drawn to illustrate the principles of the design of the multi-analyte sensor 112. In the description that follows, it is assumed that the multi-analyte sensor 112 is a MIP sensor in which exposed analyte electrodes are coated in MIPs. However, different or additional types of analyte-responsive sensors may also be included in the multi-analyte sensing device 110. For example, some or all of the exposed electrodes may have a coating including antibodies, aptamers, or any other chemical-capture agent that includes binding sites that preferentially bind to target analytes.

A MIP is a polymer material that has binding sites with a strong affinity for a target analyte. The MIP is formed by polymerizing monomers in the presence of a template (which is often the target analyte). The monomers polymerize around some or all of the template. Thus, when the template is removed, a cavity is left behind that has a size, shape, and charge distribution that corresponds to the target analyte. Thus, when the MIP is exposed to the target analyte, molecules of the target analyte tend to bind to the MIP (similar to how antibodies bind to an antigen). Generally, the higher the concentration of the target analyte, the greater the number of molecules will bind to the MIP. MIP sensors detect the concentration of the corresponding target analyte by measuring changes in electrical properties of the sensor as molecules of the target analyte bind to the binding sites. For example, the impedance of a circuit including one class of MIP decreases as the number of molecules of the target analyte bound to the MIP increases, and thus the impedance decreases as the concentration of the target analyte increases. For another class of MIP, the impedance of the circuit increases as target molecules bind to the MIP, and thus the impedance increases with the concentration of the target analyte.

The multi-analyte sensor 112 shown in FIG. 2 includes a circuit board 201, but any suitable configuration of conductors on or within an insulating substrate may be used. At one end of the circuit board 201 is an adaptor 203 that enables the multi-analyte sensor 112 to be attached (e.g., connected) to the controller 114. The adaptor 203 may clip into a socket (e.g., mounted on a printed circuit board inside the device 110 or on an exterior housing of the device) to allow relatively easy replacement of the sensor. As will be further described below, in one embodiment the adaptor 203 is a universal serial bus (USB) type-c connector otherwise referred to as a USB-C connector.

The multi-analyte sensor 112 includes a plurality of analyte electrodes 205 and a reference electrode 207. The plurality of analyte electrodes 205 are arranged in a plurality of rows 216A to 216J and columns 217A and 217B. As shown in FIG. 2, a first column 217A of analyte electrodes 205 is disposed at a first side (e.g., the left side) of the reference electrode 207 and a second column 217B of analyte electrodes 205 is disposed at a second side (e.g., the right side) of the reference electrode 207. In one embodiment, the distance D (e.g., a horizontal distance) from a center of each analyte electrode 205 to a center of the reference electrode 207 is the same. Each row 216 of analyte electrodes includes a set of analyte electrodes 205 where the set includes multiple analyte electrodes. In one embodiment, each row 216 of analyte electrodes includes at least two analyte electrodes as shown in FIG. 2. However, each row 216 may include greater or fewer analyte electrodes in other embodiments.

Each analyte electrode 205 is coated with a MIP for a target analyte. In one embodiment, each analyte electrode 205 is configured to detect a different analyte. Thus, each analyte electrode 205 may be coated with a different MIP. Alternatively, some or all of the analyte electrodes 205 are configured to detect the same analyte. That is, multiple analyte electrodes 205 are configured to detect the same analyte, or all of the analyte electrodes are configured to detect the same analyte. Example target analytes that can be detected using the multi-analyte sensor 112 include cortisol, dehydroepiandrosterone (DHEA), melatonin, progesterone, estrogen, testosterone, cytokines, C-reactive protein, and cholesterol, among many others. In contrast, the reference electrode 207 may have no coating or has been treated to have substantially invariant electrical properties regardless of the presence of target analytes.

The multi-analyte sensor 112 also includes a plurality of electrical lines 209 made from an electrical conductor such as copper. The electrical lines 209 extend from electrical pins in the adaptor 203 to the analyte electrodes 205. For example, the electrical lines 209 extend from the analyte electrodes 205 in the second column 217B of analyte electrodes to the pins of the adaptor 203. In one embodiment, each electrical line 209 is connected to a corresponding one of the analyte electrodes 205 in the second column 217B.

In contrast, each analyte electrodes 205 in the first column 217A of analyte electrodes 205 is connected to a corresponding via 211. In one embodiment, the opposite side of the circuit board 201 also includes a plurality of electrical lines that are electrically connected to the vias 211 shown in FIG. 2 and extend to pins in the opposite side of the adaptor 203.

Furthermore, the electrical lines 209 include one or more electrical lines that are electrically connected to the reference electrode 207 as shown in FIG. 2. In one embodiment, the adaptor includes a plurality of pins (e.g., 2 pins) that are electrically connected to the reference electrode 207 through two electrical lines. By using two pins to connect to the reference electrode 207, the multi-analyte sensor 212 cannot be connected in a wrong position (e.g., upside down).

In on embodiment, a single measurement is made using analyte electrodes 205 include in a single row 216 (e.g., using one or more masks as will be described below). As shown in FIG. 2, each row of analyte electrodes 216A to 216J includes a pair of analyte electrodes 205. Consequently, the measurement provides a measure of target-analytes for which the analyte electrodes 205 in the corresponding row 216 340 is coated in the MIP. Thus, different measurements can include values for the concentrations of different subsets of the target analytes sensed using the analyte electrodes 205 in the row 216.

If the analyte electrodes 205 of the multi-analyte sensor 112 are exposed to a sample, changes in the sensor's electrical properties may be used to detect the concentration of the target analytes in the sample. The target analytes selectively bond to the corresponding MIPs in each analyte electrode 205, which in turn change the electrical properties of the circuits formed by the corresponding electrical lines 209 and the reference line 207 (or lines). For example, as the concentration of a target analyte increases, a greater number of target analyte molecules bind to the MIP for that analyte, and the impedance between a corresponding electrical line 209 and reference line 207 decreases. The multi-analyte sensor device 110 may be calibrated to convert measured impedance values from the multi-analyte sensor 112 into concentrations. Additionally or alternatively, variations in the capacitance, inductance, resistance, or any other electrical property may be measured and calibrated to provide a measure of the concentration of a target analyte in the sample.

### Masks

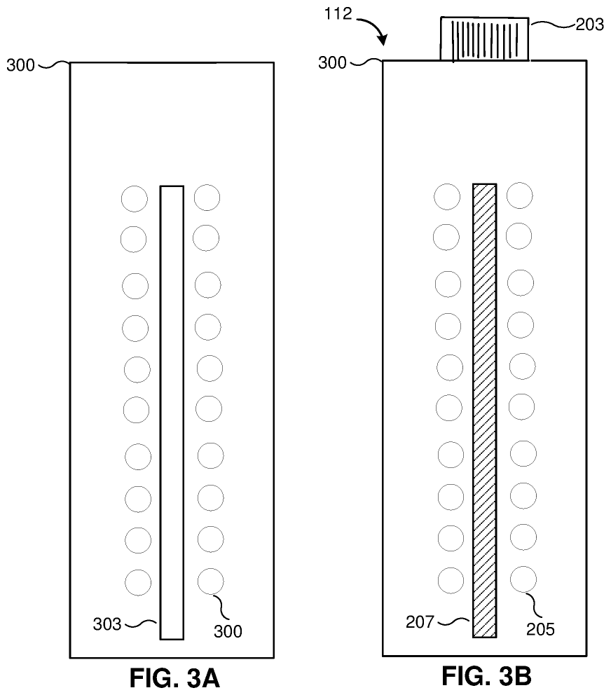

FIG. 3A illustrates one embodiment of a mask 300 configured for a single use of the multi-analyte sensor 112. In one embodiment, a “single use” of the multi-analyte sensor 112 allows for only a single exposure of the sensor 112 to a sample before the sensor 112 is replaced with an unused sensor 112. The mask 300 for a single use multi-analyte sensor 112 includes a plurality of first openings 300 and a second opening 303 that respectively expose all of the analyte electrodes 205 and reference electrode 207 while covering the electrical lines 209 and the vias 211. By exposing all of the plurality of analyte electrodes 205 and the reference electrode 207 of the multi-analyte sensor 112 using the mask 300, the multi-analyte sensor 112 is configured as a single-use (e.g., one-time use) sensor in one embodiment. That is, mask 300 allows for all of the analyte electrodes 205 in the multi-analyte sensor 112 to be used at the same time to sense one or more analytes such that the multi-analyte sensor 112 is used only once before being replaced by a new multi-analyte sensor 112.

The position of the plurality of openings 300 in the single use mask 300 corresponds to the position of the plurality of analytes electrodes 205 included in the multi-analyte sensor 112. That is, each opening 300 is aligned with a corresponding one of the analyte electrodes 205 while the mask 300 is disposed on the multi-analyte sensor 112. Similarly, the position of the second opening 303 corresponds to the position of the reference electrode 207 included in the multi-analyte sensor 112 such that the second opening 303 is aligned with the reference electrode 207 while the mask 300 is disposed on the multi-analyte sensor 112. In one embodiment, the single use mask 300 is made of a film such as polyethylene (PET), polyvinyl chloride (PVC), Kapton, or any thin polymer robust enough to be peeled off from the multi-analyte sensor 112. However, other materials such as metal may be used for the single mask 300 in other embodiments.

FIG. 3B illustrates the mask 300 placed on the multi-analyte sensor 112 according to one embodiment. As shown in FIG. 3B, the first openings 300 are aligned with the analyte electrodes 205 and the second opening 303 is aligned with the reference electrode 207 thereby exposing the analyte electrodes 205 and reference electrode 207. Thus, all of the analyte electrodes 205 and the entire reference electrode 207 are exposed to a sample (e.g., saliva) when the multi-analyte sensing device 110 is used to perform a measurement. However, as mentioned above, the mask 300 covers the electrical lines 209 and vias 211 as shown in FIG. 3B. By covering the electrical lines 209 and vias 211, the mask 300 protects the electrical lines 209 and vias 211 from deterioration due to exposure to the sample and or environment (e.g., air, water, etc.).

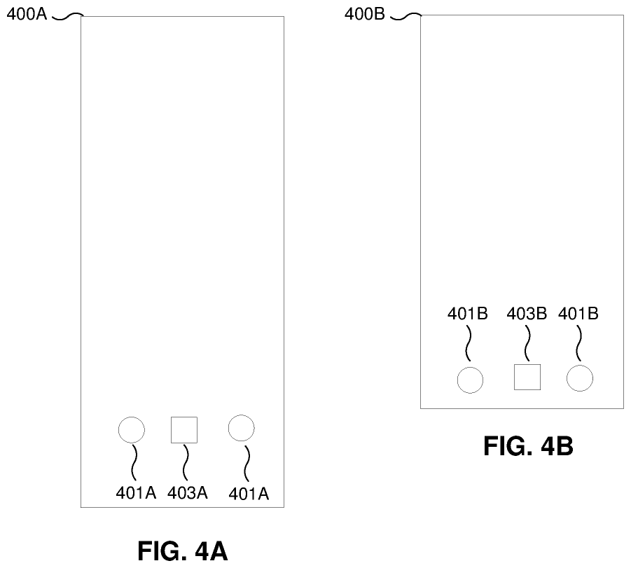

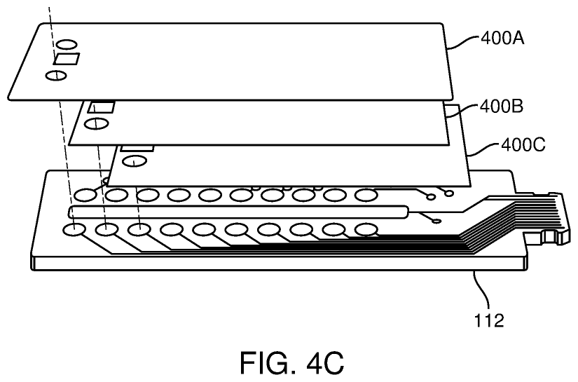

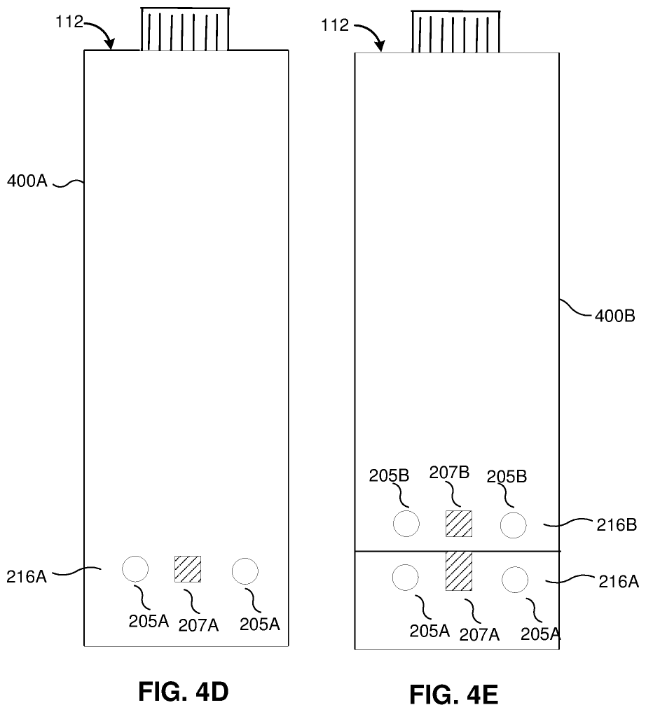

FIGS. 4A and 4B illustrate a set of masks 400 for a multi-use multi-analyte sensor 112 according to one embodiment. In one embodiment, a “multi-use” multi-analyte sensor 112 allows for multiple exposures of the sensor 112 to multiple samples at different instances in time before the sensor 112 is replaced with an unused sensor 112. In one embodiment, the set of masks 400 includes a plurality of masks 400. The plurality of masks 400 are configured to be stacked on top of each other on the multi-analyte sensor 112. Each mask 400 includes a plurality of first openings 401 and a second opening 403. However, the positions of the first openings 401 and the second opening 403 are different in each of the masks 400.

In one embodiment, each mask from the plurality of masks 400 corresponds to a particular row 216 of analyte electrodes 205 where the position of the plurality of openings 401 in each mask 400 corresponds to the position of the particular pair of analytes electrodes 205 in the particular row 216. For example, the first mask 400A may correspond to the first row 216A of analyte electrodes 205 where the openings 401A correspond to the position of the analyte electrodes 205 included in the first row 216A and the second opening 403A corresponds to the position of the portion of the reference electrode 207 disposed between the analyte electrodes 205 in the first row 216A. Similarly, the second mask 400B may correspond to the second row 216B of analyte electrodes 205 where the first openings 401B correspond to the position of the analyte electrodes 205 included in the second row 216B and the second opening 403B corresponds to the position of the portion of the reference electrode 207 disposed between the analyte electrodes 205 in the second row 216B.

In one embodiment, the masks 400 all have the same width but different lengths with the first mask corresponding to the first row of analyte electrodes (e.g., the analyte electrodes furthest from the adaptor 203) having the longest length and the last mask corresponding to the last row of analyte electrodes (e.g., the analyte electrodes closest to the adaptor 204) having the shortest length. By using masks 400 with different lengths, the amount of material required for the masks 400 is reduced. In other embodiments, the masks 400 all have the same width and length.

Although only two masks 400 are shown in FIG. 4, the multi-analyte sensor 112 may include additional masks 400. In one embodiment, the total number of masks included in the plurality of masks 400 is based on the total number of rows 216 of analyte electrodes 205 included in the multi-analyte sensor 112. For example, the total number of masks included in the plurality of masks 400 is equal to the total number of rows 216 of analyte electrodes 205. In the example herein, the masks 400 may include a total of ten masks given there are a total of 10 rows 216A to 216J of analyte electrodes 205 in the multi-analyte sensor 112. The example sensor 112 may be designed for taking one measurement a day over a ten day period for example. However, any number of masks may be used depending on the total number of rows of analyte electrodes that are included in the multi-analyte sensor 112.

By exposing only a pair of analyte electrodes 205 at a time using masks 400, the multi-analyte sensor 112 is configured as a multi-use analyte sensor in one embodiment. Each usage of the multi-analyte sensor 112 using the masks 400 exposes only a single pair of analyte electrodes 205 during the measurement of analytes. During subsequent usages of the multi-analyte sensor 112, a mask 400 is removed from the multi-analyte sensor 112 thereby exposing another mask that is disposed underneath the mask that was removed.

FIG. 4C shows a view of the multi-analyte sensor 112 using masks 400 in a stacked arrangement according to one embodiment. FIG. 4C illustrates only three masks 400A to 400C, however in practice the sensor 112 includes the same number of masks as rows 216 of analyte electrodes in one embodiment. As shown in FIG. 4C, the masks 400 are arranged in descending order based on the row 216 of analyte electrodes that is associated with a given mask. For example, mask 400C associated with the third row 216C of analytes electrodes is the first mask shown in FIG. 4C, mask 400B associated with the second row 216B of analyte electrodes is the second mask, and mask 400A associated with the first row 216A of analyte electrodes is the last mask that is placed on the multi-analyte sensor 112. As will be further described with respect to FIGS. 4D and 4E, masks are removed after each measurement to expose different rows of analyte electrodes 205.

FIG. 4D illustrates a plan view of the multi-analyte sensor 112A with all of the masks 400 disposed on the multi-analyte sensor 112. However, only the first mask 400A from the plurality of masks 400 is visible as the remaining masks are disposed underneath the first mask 400A. As shown in FIG. 4D, the first mask 400A exposes only the analyte electrodes 205A includes in the first row 216A of analyte electrodes 205 and the portion 207A of the reference electrode 207 disposed between the analyte electrodes 205A. In one embodiment, the multi-analyte sensing device 110 takes a first measurement of analytes of a first sample using the exposed analyte electrodes 205A and exposed portion 207A of the reference electrode 207.

At a later time (e.g., the next day), a second measurement of analytes from a second sample may be required. Accordingly, the first mask 400A may be removed by peeling off the first mask 400A for example. The first mask 400A may be discarded at that time. By removing the first mask 400A, the second mask 400B is exposed as shown in FIG. 4E. The second mask 400B exposes the analyte electrodes 205B and the portion 207B of the reference electrode 207 in the second row 216B. The multi-analyte sensing device 110 takes a second measurement of analytes of a second sample using the exposed analyte electrodes 205B and exposed portion 207B of the reference electrode 207. As shown in FIG. 4E, by removing the first mask 400A, electrodes 205A and the portion 207A of the reference electrode 207 are still exposed. In one embodiment, the multi-analyte sensing device 110 may ignore any measurements received from the electrodes 205A. The process of removing the masks and taking subsequent measurements is repeated until a last measurement is performed using the last mask in the set of masks 400.

### Sensor Connection

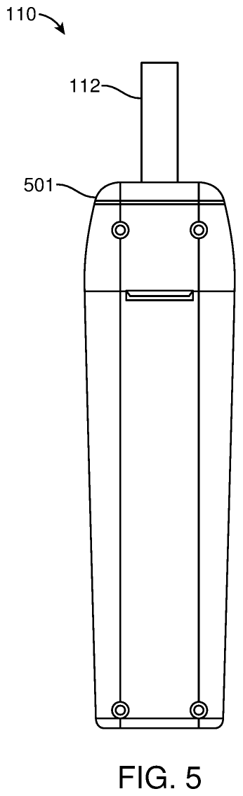

FIG. 5 illustrates one embodiment of the multi-analyte sensing device 110. As shown in FIG. 5, the multi-analyte sensing device 110 is a handheld device. The multi-analyte sensing device 110 includes the multi-analyte sensor 112 as previously described above and a case 501. The case 501 is configured to be held in a person's hand and is used by a user to expose the sensor 112 to a sample. For example, the user may grip the case 501 and direct the sensor 112 into the user's mouth to expose the sensor 112 to saliva. The case 501 may be made of material such as plastic, but other materials may be used.

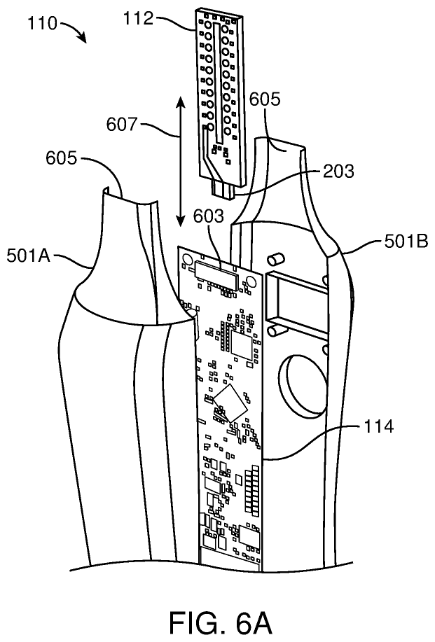

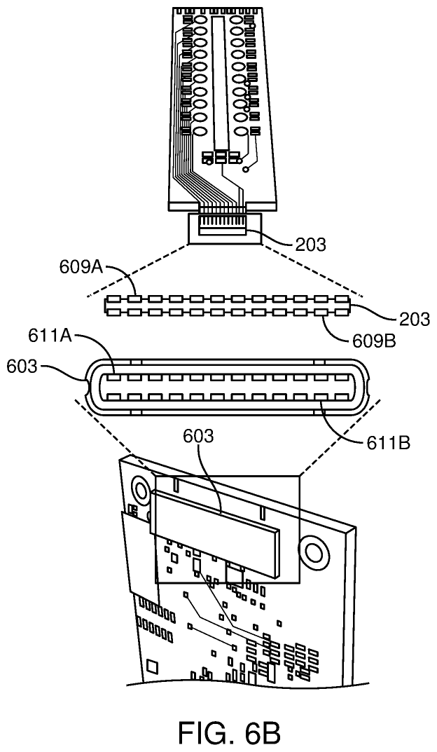

FIG. 6A illustrates an exploded view of the multi-analyte sensing device 110 according to one embodiment. The multi-analyte sensing device 110 includes the multi-analyte sensor 112, the case 501, and the controller 114 housed within the case 501. As shown in FIG. 6A, the case 501 includes a first case part 501A and a second case part 501B that join together to form the case 501. The first case part 501A and the second case part 501B may be attached to each other using fasteners (e.g., screws) for example.

In one embodiment, the case 501 includes an insertion point 605 through which the sensor 112 is inserted in order to connect the sensor 112 to a connector 603 of the controller 114. The insertion point 605 is an opening formed at one end of the case 501 when both case parts 501A and 501B are attached together. The opening has a size large enough for the sensor 112 to be inserted into in order to connect the sensor 112 to the connector 603 of the controller 114.

In one embodiment, the multi-analyte sensor 112 is removable such that the user of the multi-analyte sensing device 110 can disconnect the sensor 112 from the controller 114 by pulling the sensor 112 away from the case 501 in a direction 607 along the length of the case 501. The multi-analyte sensor 112 may be disconnected from the controller 112 in order for the user to remove a mask from the sensor 112 as previously described above, for example. In another example, the multi-analyte sensor 112 may be disconnected from the controller 112 in order to replace the multi-analyte sensor 112 with another multi-analyte sensor. As described above, a multi-analyte sensor 112 has a finite number of uses before needing to be replaced. The user may remove the multi-analyte sensor 112 from the device 110 and replace the used sensor 112 with another unused multi-analyte sensor 112 for example.

FIG. 6B illustrates a detailed view of the adaptor 203 and connector 603 according to one embodiment. Generally, the connector 603 receives the impedance values from the adaptor 203 of the multi-analyte sensor 112 and the controller 114 determines concentrations of analytes based on the received impedance values as previously described above. In one embodiment, the adaptor 204 and connector 603 are USB-Type C connectors. Specifically, the adaptor 203 on the multi-analyte sensor 112 is a female USB-Type C connector and the connector 603 is a male USB-Type C connector in one embodiment.

As shown in FIG. 6B, the adaptor 203 includes a first set of pins 609A on a first side of the adaptor 203 and a second set of pins 609B on a second side of the adaptor 203 that is opposite the first side. The first set of pins 609A are connected to the electrical lines 209 extending from the second column 217B of analyte electrodes shown in FIG. 2 and the second set of pins 609A are connected to the electrical lines on the opposite side of the sensor 112 that are electrically connected to the first column 217A of analyte electrodes in FIG. 2.

Similarly, the connector 603 includes a first set of pins 611A on a first side of the connector 603 and a second set of pins 611B on a second side of the connector 603 that is opposite the first side. The first set of pins 611A are configured to connect to the first set of pins 609A of the adaptor 203 and the second set of pins 611B are configured to connect to the second set of pins 609B on the adaptor. In one embodiment, each pin from the first set of pins 611A is configured to connect to a corresponding pin from the first set of pins 609A and each pin from the second set of pins 611B is configured to connect to a corresponding pin from the second set of pins 609B.

### Water Proofing

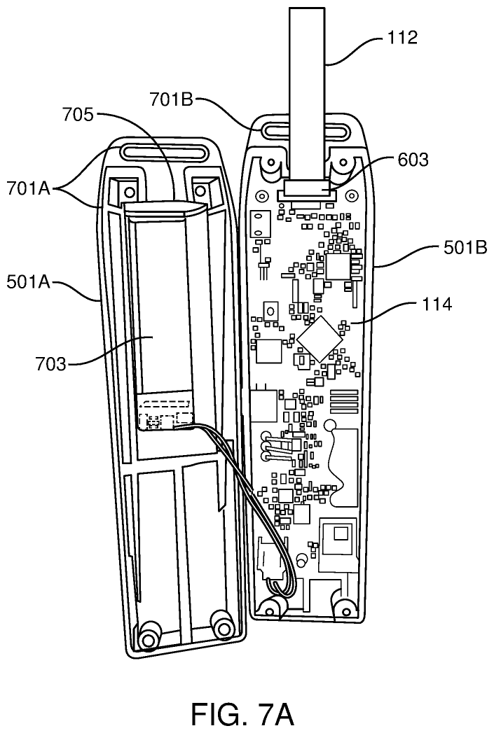

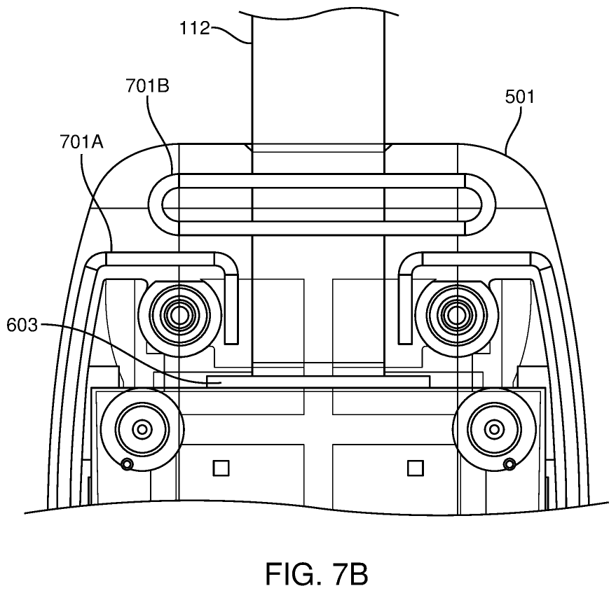

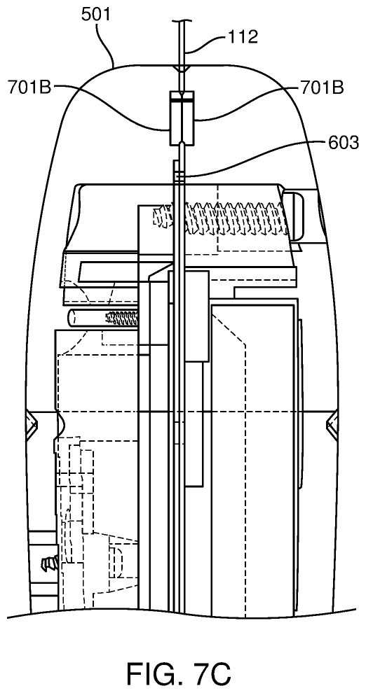

The samples used for measuring of analyte concentrations in the samples may be made of material (e.g., saliva) that may damage components of the device 110 upon contact of the components. In one embodiment, the case 501 of the multi-analyte sensing device 110 includes one or more sealing mechanisms to form a watertight seal that prevents or at least reduces exposure of the components of the device 110 to the sample and the environment. In one embodiment, the sealing mechanisms are gaskets, but other sealing mechanisms may be used. FIG. 7A illustrates the internal components of the device 110 including the controller 114 and a battery 703.

In one embodiment, the case 501 includes a plurality of grooves in which the one or more gaskets 701 are disposed. As shown in FIGS. 7A to 7C, the gaskets 701 may include a first gasket 701A disposed along the perimeter of the case 501. The first gasket 701A may be disposed in a groove formed along the perimeter of the case 501. In one embodiment, the ends of the first gasket 701A are not connected to each other in the area 705 where the connector 603 is disposed. Thus, the first gasket 701A does not form a watertight seal in the area 705 of the connector 603.

The gaskets 701 may also include a second gasket 701B that surrounds the opening 605 of the case through which the sensor 112 is inserted into the connector 603 to form the watertight seal in the area 705 of the connector 603. In one embodiment, each half of the case 501A and 501B includes a second gasket 701B. Thus, the device 110 includes at least two second gaskets 701B. For example, the second gasket 701B may include a first second gasket disposed at a position on the first case part 501A that is aligned with but non-overlapping with the connector 603, and the second case part 501B includes a second gasket disposed at a position on the second case part 501B that is aligned with but non-overlapping with the connector 603.

When both halves of the cases 50A and 501B are attached together, the two second gaskets 701B contact each other to form a watertight seal in the area 705 thereby protecting the connector 603 from the sample. That is, the second gaskets 701B prevent or at least reduce the connector 603 from exposure to the sample. In one embodiment, an opening is formed between the second gaskets 701B having a size that is large enough for the sensor 112 to pass through in order to connect to the connector 603 while reducing the amount of sample that may be exposed to the connector 603, if any. Thus, a portion of the sensor 112 is between the pair of second gaskets 701B while the sensor 112 is connected to the controller 114 via the connector 603. As shown in FIG. 7B, the second gaskets 701B have an oval shape, but other shapes may be used for the second gaskets 701B.

### Calibration

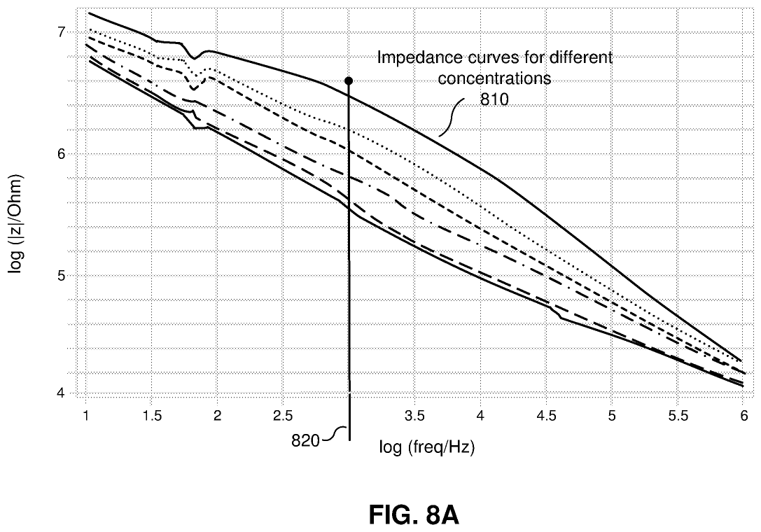

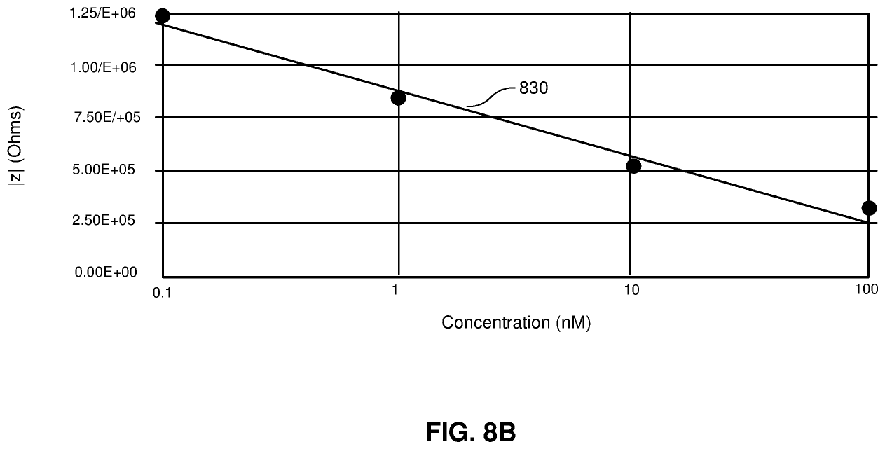

FIGS. 8A and 8B illustrate an example approach for calibrating the multi-analyte sensor 112. In a calibration phase of the device 110, the variation of one or more electrical parameters of the sensor 112 with frequency is measured for a range of samples with known concentrations of a target analyte are measured. For example, in FIG. 8A, a set of six impedance curves 810 corresponding to six samples with differing concentrations of the target analyte are shown. Based on the log-log plot of impedance against frequency, a sampling frequency 820 is selected. The sampling frequency 820 may be selected using one or more criteria. For example, in one embodiment, the frequency with the largest impedance range is selected as the sampling frequency 820 to provide a good signal-to-noise ratio. In another embodiment, the frequency with the most linear response across the range of concentrations of interest for the target analyte or analytes is selected. Different sampling frequencies 520 may be selected for different analytes.

FIG. 8B is a plot of impedance against concentration of the target analyte at the target frequency, according to one embodiment. In FIG. 8B, a linear trend line 830 is fitted to the data points. Thus, in a measurement phase, the impedance measured for the electrical line or lines of the sensor 112 corresponding to the target analyte can be converted to a concentration of the target analyte using the trend line 830. Use of a linear trend line 830 has the advantage of simplicity, making it easy and computationally inexpensive to convert any measurement of impedance into the corresponding concentration. However, in other embodiments, non-linear calibration may be used, such as fitting a higher-order polynomial to the measured data points or using a lookup table with piecewise interpolation between the measured data points.

Although FIGS. 8A and 8B illustrate the calibration technique using impedance, a similar approach can be adopted for any electrical parameter of the sensor 112. The parameter may be measured for a range of concentrations at a range of frequencies to identify a sampling frequency and then a mapping (e.g., a trend line or lookup table) can be determined between the parameter and the concentration of the target analyte at the sampling frequency.

### Additional Considerations

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the element or component is present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs for a multi-analyte sensor as well as methods for making and using such a sensor. Thus, while particular embodiments and applications have been illustrated and described, it is to be understood that the described subject matter is not limited to the precise construction and components disclosed. The scope of protection should be limited only by the following claims.

What is claimed is:

1. A multi-analyte device comprising:
a case having an opening at one end of the case;
a replaceable sensor having a first part disposed outside of the case and a second part disposed within the case through the opening, the first part of the replaceable sensor comprising a plurality of electrodes coated with one or more different molecular imprinted polymer (MIP) coatings where an electrical property of the one or more different MIP coatings changes responsive to exposure to one or more target analytes; and
a controller housed within the case and attached to the second part of the replaceable sensor, the controller including circuitry that receives one or more signals from the replaceable sensor that are indicative of the changes to the electrical properties of the plurality of electrodes and determines a concentration of the one or more target analytes based on the one or more signals,
wherein the replaceable sensor is removably attachable to the controller.

2. The multi-analyte device of claim 1, wherein the second part of the replaceable sensor lacks any electrodes coated with MIP coatings.

3. The multi-analyte device of claim 1, wherein the second part of the replaceable sensor comprises an adaptor, and the controller comprises a connector, the adaptor configured to be inserted into the connector.

4. The multi-analyte device of claim 3, wherein the adaptor comprises a female universal serial bus (USB) type C connector and the connector comprises a male USB type c connector.

5. The multi-analyte device of claim 1, wherein the plurality of electrodes are arranged on the first part of the replaceable sensor in a plurality of rows, each of the plurality of rows including at least two electrodes from the plurality of electrodes.

6. The multi-analyte device of claim 5, wherein the replaceable sensor further comprises:
a reference electrode disposed between the at least two electrodes from each of the plurality of rows.

7. The multi-analyte device of claim 6, wherein a first electrode from the at least two electrodes from each of the plurality of rows is at a first side of the reference electrode and a second electrode from the at least two electrodes from each of the plurality of rows is at a second side of the reference electrode, the second side opposite the first side.

8. The multi-analyte device of claim 7, wherein a first distance from a center of the first electrode from each of the plurality of rows to a center of the reference electrode is the same as a second distance from a center of the second electrode from each of the plurality of rows to the center of the reference electrode.

9. The multi-analyte device of claim 6, wherein the replaceable sensor further comprises:
a mask disposed on the first part of the replaceable sensor, the mask including a plurality of first openings and a second opening,
wherein a position of each of the plurality of first openings is aligned with a position of a corresponding one of the plurality of electrodes such that the corresponding one of the plurality of electrodes is exposed through the corresponding first opening, and a position of the second opening is aligned with a position of the reference electrode such that the reference electrode is exposed through the second opening.

10. The multi-analyte device of claim 9, wherein the replaceable sensor having the mask is configured to be a single use sensor and the controller is configured to determine the concentration of the one or more target analytes using all of the plurality of electrodes that are exposed to a single sample of the one or more target analytes.

11. The multi-analyte device of claim 6, wherein the replaceable sensor further comprises:
a plurality of masks disposed on the first part of the replaceable sensor, each of the plurality of masks associated with a corresponding row of the plurality of rows of electrodes.

12. The multi-analyte device of claim 11, wherein each of the plurality of masks includes a plurality of first openings and a second opening,
wherein positions of the plurality of first openings of each mask are aligned with positions of at least two electrodes included in the corresponding row, and a position of the second opening corresponds to a position of a portion of the reference electrode disposed between the at least two electrodes.

13. The multi-analyte device of claim 12, wherein each of the plurality of masks exposes the at least two electrodes included in the corresponding row and the portion of the reference electrode disposed between the at least two electrodes, and remaining electrodes from the plurality of electrodes and a remaining portion of the reference electrode are not exposed by the mask.

14. The multi-analyte device of claim 13, wherein each of the plurality of masks is configured to be removed from the replaceable sensor in a predetermined order thereby exposing different rows of the plurality of electrodes, wherein a removal of one of the plurality of masks exposes another mask from the plurality of masks that is disposed under the removed one of the plurality of masks.

15. The multi-analyte device of claim 14, wherein the replaceable sensor is configured as a multi-use sensor and the controller is configured to determine the concentration of the one or more target analytes using rows of the plurality of electrodes that are exposed to a plurality of different samples of the one or more target analytes at different instances of time.

16. The multi-analyte device of claim 13, wherein the plurality of masks have a same width but each mask from the plurality of masks have a different length.

17. The multi-analyte device of claim 13, wherein the plurality of masks have a same width and a same length.

18. The multi-analyte device of claim 1, wherein an interior surface of the case includes a gasket around a perimeter of the case.

19. The multi-analyte device of claim 3, wherein the case comprises a first case part and a second case part configured to be attached to the first case part.

20. The multi-analyte device of claim 19, wherein the first case part includes a first gasket disposed at a position on the first case part that is aligned with but non-overlapping with the connector, and the second case part includes a second gasket disposed at a position on the second case part that is aligned with but non-overlapping with the connector.

21. The multi-analyte device of claim 20, wherein the first gasket and the second gasket are configured to contact with each other while the first case part and the second case part are attached to each other, and a portion of the replaceable sensor is disposed between the first gasket and the second gasket while the adaptor of the replaceable sensor is attached to the connector.

22. The multi-analyte sensor of claim 1, wherein the one or more target analytes include cortisol, dehydroepiandrosterone (DHEA), melatonin, progesterone, estrogen, testosterone, C-reactive protein, or cholesterol.

23. A multi-analyte device comprising:
a case having an opening; and
a controller housed within the case, the controller including a connector configured to receive a replaceable sensor including a plurality of electrodes coated with one or more different molecular imprinted polymer (MIP) coatings where an electrical property of the one or more different MIP coatings changes responsive to exposure to one or more target analytes.

24. The multi-analyte device of claim 23, wherein the controller is configured to receive one or more signals from the replaceable signal that are indicative of the changes to the electrical properties of the plurality of electrodes when the replaceable sensor is received by the connector and is exposed to the one or more target analytes, and is configured to determine a concentration of the one or more target analytes based on the one or more signals.

25. A multi-analyte device comprising:
a case having an opening;
a controller housed within the case; and
a replaceable sensor configured to be inserted into the opening of the case to connect the replaceable sensor to the controller, the replaceable sensor comprising a plurality of electrodes coated with one or more different molecular imprinted polymer (MIP) coatings where an electrical property of the one or more different MIP coatings changes responsive to an exposure of the replaceable sensor to one or more target analytes.
